(19) United States
(12) Patent Application Publication
(10) Pub. No.: US 2017/0319268 A1
(43) Pub. Date: Nov. 9, 2017

(54) INSERTION INSTRUMENT AND MEDICAL TREATMENT SYSTEM

(71) Applicant: OLYMPUS CORPORATION, Tokyo (JP)

(72) Inventor: Tsunetaka AKAGANE, Hachioji-shi (JP)

(73) Assignee: OLYMPUS CORPORATION, Tokyo (JP)

(21) Appl. No.: 15/656,346

(22) Filed: Jul. 21, 2017

Related U.S. Application Data
(63) Continuation of application No. PCT/JP2016/054122, filed on Feb. 12, 2016.

(30) Foreign Application Priority Data
Mar. 4, 2015 (JP) 2015-042935

(51) Int. Cl. A61B 18/14; A61B 1/00; A61B 18/12; A61M 13/00; A61B 1/015; A61B 1/313; A61B 18/00

(52) U.S. Cl. CPC A61B 18/1482; A61B 1/015; A61B 1/00006; A61B 18/1206; A61B 1/3132; A61M 13/006; A61B 1/00154; A61B 1/00096; A61B 2018/00702; A61B 2018/00994

(57) ABSTRACT

An insertion instrument includes a shaft projecting, by being inserted through a hole of a fixing instrument, from a distal end of the fixing instrument toward a distal side, and a gas supply channel formed in the shaft section and having a jet port, which jets a gas, in a distal portion of the shaft section. The gas is supplied from a proximal side to the distal side toward the jet port in the gas supply channel, and an extension dimension along the longitudinal axis from the jet port toward the proximal side is greater than an extension dimension of the hole of the fixing instrument.

### CROSS-REFERENCE TO RELATED APPLICATIONS

This is a Continuation Application of PCT Application No. PCT/JP2016/054122, filed Feb. 12, 2016 and based upon and claiming the benefit of priority from prior Japanese Patent Application No. 2015-042935, filed Mar. 4, 2015, the entire contents of which are incorporated herein by reference.

### BACKGROUND OF THE INVENTION

### 1. Field of the Invention

The present invention relates to an insertion instrument which is inserted into a body cavity, and a medical treatment system which includes the insertion instrument and is configured such that a gas is supplied into the body cavity from the outside of the body.

### 2. Description of the Related Art

Jpn. Pat. Appln. KOKAI Publication No. 2000-175931 discloses a medical treatment system including an endoscope and an energy treatment instrument which are inserted into an abdominal cavity that is a body cavity. In this medical treatment system, two trocars (fixing instruments) are fixed to a body wall, and each of an insertion section (shaft section) of the endoscope (insertion instrument) and the energy treatment instrument is inserted into the abdominal cavity through a hole of the corresponding trocar. A distal portion of the energy treatment instrument is provided with a treatment section, and the treatment section treats a treated target in the body cavity by using supplied high-frequency electric power (energy). In addition, a distal portion of the insertion section of the endoscope is provided with an imaging element. In the abdominal cavity, the imaging element is disposed near the treatment section, and the imaging element captures a subject in a treated region by the treatment section and a vicinity thereof. Besides, a gas supply channel, which supplies carbon dioxide (gas) into the abdominal cavity from the outside of the body, is formed between the trocar, through which the insertion section of the endoscope is inserted, and the insertion section. The carbon dioxide, which is supplied through the gas supply channel, is jetted into the abdominal cavity from the distal end of the trocar.

### BRIEF SUMMARY OF THE INVENTION

According to one aspect of the invention, an insertion instrument which is inserted into a body cavity by being inserted through a hole of a fixing instrument in a medical treatment system, the medical treatment system including the fixing instrument configured to form the hole which establishes communication between an outside of a body and the body cavity, and configured to be fixed to a body wall, and including a treatment section configured to perform a treatment by using energy in the body cavity, the insertion instrument including: a shaft section which extends along a longitudinal axis from a proximal portion toward a distal

portion, and projects, by being inserted through the hole of the fixing instrument, from a distal end of the fixing instrument toward a distal side in the body cavity; and a gas supply channel which is formed in the shaft section and has a jet port, which jets a gas, in the distal portion of the shaft section, the gas supply channel being configured such that the gas is supplied from a proximal side to the distal side toward the jet port, and configured such that an extension dimension along the longitudinal axis from the jet port toward the proximal side is greater than an extension dimension of the hole of the fixing instrument.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view illustrating a medical treatment system according to a first embodiment,

FIG. 2 is a schematic view illustrating a medical treatment system according to a first modification,

FIG. 3 is a schematic view illustrating a medical treatment system according to a second modification, and

FIG. 4 is a schematic view illustrating a medical treatment system according to a third modification.

### DETAILED DESCRIPTION OF THE INVENTION

### First Embodiment

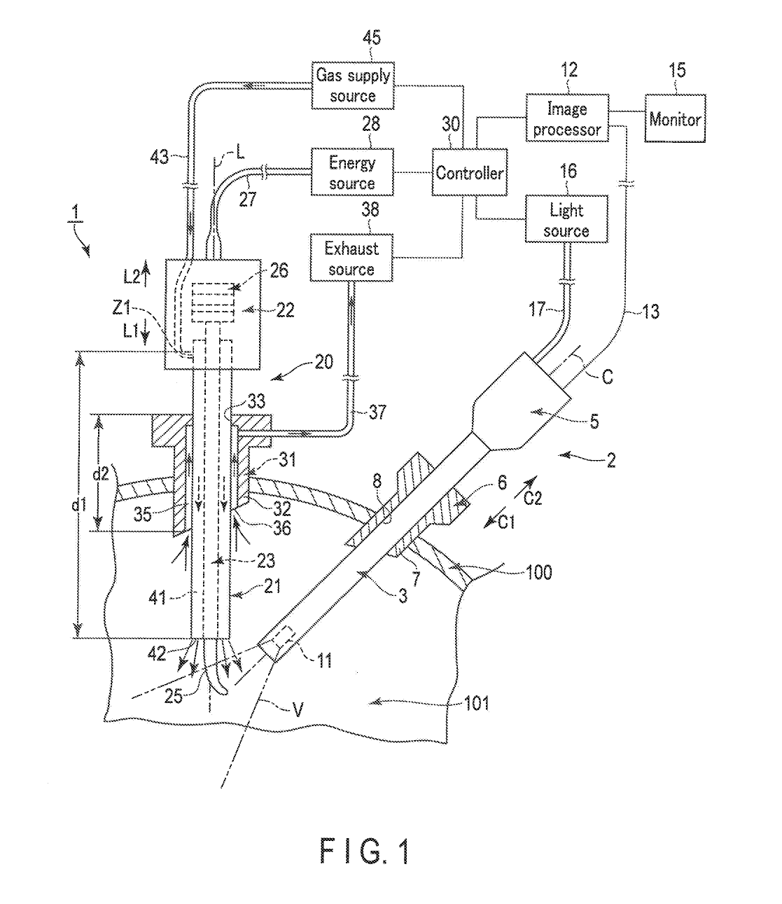

A first embodiment of the present invention will be described with reference to FIG. 1.

FIG. 1 is a view illustrating a medical treatment system 1 of the present embodiment. As illustrated in FIG. 1, the medical treatment. system 1 includes an endoscope (rigid endoscope) 2 which is an insertion member. The endoscope 2 has a longitudinal axis (endoscope longitudinal axis) C. Here, one side of a direction parallel to the longitudinal axis C of the endoscope 2 is a distal side (an arrow C1 side in FIG. 1) of the endoscope 2, and a side opposite to the distal side is a proximal side (an arrow C2 side in FIG. 1) of the endoscope 2. The endoscope 2 includes an insertion section (endoscope insertion section) 3 which extends along the longitudinal axis C, and an operation section (endoscope operation section) 5 which is provided on the proximal side with respect to the insertion section 3. The insertion section 3 has at distal portion and a proximal portion, and a distal end of the endoscope 2 is formed by a distal end of the insertion section 3.

In addition, the medical treatment system 1 includes a trocar (first trocar) 6 which is a fixing instrument. The trocar 6 includes a piercing portion (first piercing portion) 7. By the piercing portion 7 being pierced into a body wall 100, the trocar 6 is fixed to the body wall 100. Besides, a hole (first hole) 8, through which the insertion section 3 of the endoscope 2 or the like is passed, is formed in the trocar 6. In the state in which the trocar 6 is fixed to the body wall 100, the outside of the body (external environment) and a body cavity 101 communicate with each other through the hole 8. The insertion section 3 of the endoscope 2 is inserted through the hole 8 of the trocar 6 from the distal side, and is inserted into the body cavity 101. In the state in which the insertion section 3 is inserted into the body cavity 101, the distal portion of the insertion section 3 is located on the distal side (distal direction side) with respect to a distal end of the trocar 6 (a distal end of the piercing portion 7). Specifically, the insertion section 3 projects from the distal end of the trocar 6 toward the distal side. In addition, the operation section 5 is located in the outside of the body. Here, the body cavity is, for example, an abdominal cavity, a thoracic cavity, a pelvic cavity, or a perinephric cavity. However, a lumen cavity, which extends in the inside of the esophagus, the inside of the large intestine, or the like, is not included in the body cavities.

In the meantime, in the state in which the insertion section 3 of the endoscope 2 is inserted through the hole 8 of the trocar 6, airtightness is kept between the insertion section 3 of the endoscope 2 and the trocar 6 at a proximal end of the hole 8, for example, by using a publicly known valve mechanism. Thus, when pneumoperitoneum was performed by using, for example, carbon dioxide, the carbon dioxide is prevented from flowing out through the proximal end of the hole 8 of the trocar 6.

An imaging element 11, such as a CCD, is provided in the distal portion of the insertion section 3. In addition, the medical treatment system 1 includes an image processor 12 as an image processing device, which is disposed in the outside of the body. One end of an imaging cable 13 is connected to the imaging element 11. The imaging cable 13 extends through the inside of the insertion section 3 and the inside of the operation section 5, and the other end of the imaging cable 13 is connected to the image processor 12. In addition, the image processor 12 is electrically connected to a monitor 15 which is a display device disposed in the outside of the body. The imaging element 11 captures a subject in a view field range V of the body cavity 101 through an observation window (not shown) which is provided on a distal surface of the insertion section 3. By the image capturing being executed by the imaging element 11, an imaging signal (electric signal) is transmitted to the image processor 12 via the imaging cable 13, and is subjected to image processing by the image processor 12. Then, the image-processed subject image is displayed on the monitor 15.

The medical treatment system 1 includes a light source 16 such as a lamp, which is disposed in the outside of the body. A light guide 17 is optically connected to the light source 16. The light guide 17 extends to the distal portion of the insertion section 3 through the inside of the operation section 5 and the inside of the insertion section 3. Light, which is emitted from the light source 16, is guided through the light guide 17. Then, the light is radiated to the view field range V of the imaging element 11 from one end (distal end) of the light guide 17 through an illumination window (not shown) which is provided on the distal surface of the insertion section 3.

In addition, the medical treatment system 1 includes an energy treatment instrument 20 which is an insertion instrument. In the present embodiment, the energy treatment instrument 20 is a treatment instrument which performs a treatment by using high-frequency electric power and ultrasonic vibration as energy. The energy treatment instrument 20 has a longitudinal axis (treatment instrument longitudinal axis) L. Here, one side of a direction parallel to the longitudinal axis L of the energy treatment instrument 20 is a distal side (an arrow L1 side in FIG. 1) of the energy treatment instrument 20, and a side opposite to the distal side is a proximal side (an arrow L2 side in FIG. 1) of the energy treatment instrument 20. The energy treatment instrument 20 includes a sheath 21 which is a shaft section extending along the longitudinal axis L, and a held section 22 which can be held and is coupled to a proximal side of the sheath 21. The sheath 21, which has a cylindrical shape, has a distal portion and a proximal portion.

In addition, the energy treatment instrument 20 includes a transmitting member (probe) 23 which can transmit ultrasonic vibration. The transmitting member 23 is inserted through the sheath 21, and extends from the inside of the held, section 22 toward the distal side (distal direction) through the inside of the sheath 21. A distal portion of the transmitting member 23 (a distal portion of the energy treatment instrument 20) is provided with a treatment section 25. The treatment section 25 of the transmitting member 23 projects from a distal end of the sheath 21 toward the distal side, and a distal end of the energy treatment instrument 20 is formed by a distal end of the treatment section 25 (a distal end of the transmitting member 23). In the present embodiment, the treatment section 25 is formed in a spatula shape. In addition, an ultrasonic transducer 26, which is a vibration generator, is provided in the inside of the held section 22, and the ultrasonic transducer 26 is coupled to a proximal portion of the transmitting member 23.

The medical treatment system 1 includes a trocar (second trocar) 31 as a fixing instrument, separately from the trocar (first trocar) 6. The trocar 31 includes a piercing portion (second piercing portion) 32. By the piercing portion 32 being pierced into the body wall 100, the trocar 31 is fixed to the body wall 100. Besides, a hole (second hole) 33, through which the sheath 21 of the treatment instrument 20 and the transmitting member 23 are inserted, is formed in the trocar 31. In the state in which the trocar 31 is fixed to the body wall 100, the outside of the body (external environment) and the body cavity 101 communicate with each other through the hole 33. The sheath 21 of the treatment instrument 20 and the transmitting member 23 are inserted through the hole 33 of the trocar 31 from the distal side, and are inserted into the body cavity 101. In the state in which the sheath 21 and the transmitting member 23 are inserted into the body cavity 101, the distal portion of the sheath 21 and the treatment section 25 of the transmitting member 23 are located on the distal side (distal direction side) with respect to a distal end of the trocar 31 (a distal end of the piercing portion 32). Specifically, the sheath 21 projects from the distal end of the trocar 31 toward the distal side. In addition, the held section 22 is located in the outside of the body.

In the medical treatment system 1, one end of a cable 27 is connected to the held section 22. In addition, the other end of the cable 27 is connected to an energy source 28. The energy source 28 includes, for example, an electric power source, a converter circuit which converts electric power from the electric power source to electric power for generating ultrasonic vibration, and a converter circuit which converts electric power from the electric power source to high-frequency electric power. The electric power for generating ultrasonic vibration, (ultrasonic electric energy) is supplied from the energy source 28 to the ultrasonic transducer 26 via an electric wiring line (not shown) which extends through the inside of the cable 27. Thereby, the ultrasonic transducer 26 generates ultrasonic vibration, and the generated ultrasonic vibration is transmitted to the treatment section 25 via the transmitting member 23. The high-frequency electric power (high-frequency electric energy) from the energy source is supplied to the treatment section 25 and is also supplied to an electrode plate (not shown) which is disposed in the outside of the body. In the state in which the high-frequency electric power is supplied to the treatment section 25, the treatment section 25 is put in contact with the treated target, and thereby a high-frequency current flows through the treated target between the treatment section 25 and the electrode plate. Accordingly, the ultrasonic vibration and high-frequency electric power are supplied as energy to the treatment section 25 which is provided in the distal portion of the energy treatment instrument (insertion instrument) 20, and the treatment section 25 performs a treatment by using the supplied energy.

The medical treatment system 1 includes a controller 30 which controls the entirety of the system. The controller 30 includes a processor including a CPU (Central Processing Unit) or an ASIC (application specific integrated circuit), and a storage such as a memory. The controller 30 detects a processing state of images in the image processor 12, an emission state of light from the light source 16, and a supply state of energy from the energy source 28, and controls an image process in the image processor 12, the emission of light from the light source 16, and the supply of energy from the energy source 28. For example, by an operation input being executed by an energy operation button (not shown) provided in the held section 22, the controller 30 controls the energy source 28, thereby causing the energy source 28 to supply electric power, which generates ultrasonic vibration, to the ultrasonic transducer 26, and causing the energy source 28 to supply high-frequency electric power to the treatment section 25 and the electrode plate. In addition, in one example, the energy source 28 and controller 30 may be configured as an integral energy control device.

In the state in which the sheath 21 and transmitting member 23 of the treatment, instrument 20 are inserted through the hole 33 of the trocar 31, an exhaust channel 35 is formed by the hole 33 between an outer peripheral surface of the sheath 21 and an inner peripheral surface of the trocar 31. In the state in which the sheath 21 of the treatment instrument 20 is inserted through the hole 33 of the trocar 31, the exhaust channel 35 extends in the inside of the trocar 31 along the longitudinal axis L of the sheath 21. In addition, in the state in which the trocar 31 is fixed to the body wall 100, the exhaust channel 35 opens to the body cavity 101 in a suction port 36 which is formed at a distal end of the hole 33. One end of an exhaust tube 37 is connected to the trocar 31. The inside of the exhaust tube 37 communicates with the exhaust channel 35. The exhaust tube 37 extends to the outside of the trocar 31 in the outside of the body, and the other end of the exhaust tube 37 is connected to an exhaust source 38 which is disposed in the outside of the body. In the meantime, in the state in which the sheath 21 and transmitting member 23 are inserted through the hole 33 of the trocar 31, airtightness is kept between the sheath 21 and trocar 31 at a proximal end of the hole 33, for example, by using a publicly known valve mechanism. Thus, a gas is prevented from flowing out from the exhaust, channel 35, without passing through the inside of the exhaust tube 37 or the suction port 36. Accordingly, for example, when pneumoperitoneum was performed by using, for example, carbon dioxide, the carbon dioxide is prevented from flowing out through the proximal end of the hole 33 of the trocar 31.

The exhaust source 38 includes an exhaust pump. The controller 30 detects the driving state of the exhaust pump, and controls the driving of the exhaust pump. Specifically, the controller 30 detects the state of exhaust by the exhaust source 38, and controls the exhaust by the exhaust source 38. By the exhaust pump being driven by the controller 30, the exhaust by the exhaust source 38 is performed, and a flow from the body cavity 101 (distal side) toward the outside of the body (proximal side) is formed in the exhaust channel 35. Thereby, in the suction port 36 located in the body cavity 101, a gas is sucked into the exhaust channel 35. In addition, the gas sucked from the suction port 36 is exhausted to the outside of the body through the exhaust channel 35 and the inside of the exhaust tube 37. Thereby, the gas, which is exhausted to an exhaust tank of the exhaust source 38, is collected.

In the present embodiment, the controller 30 controls the exhaust by the exhaust source 38, based on the supply state of energy from the energy source 28 (the supply state of electric power, which generates ultrasonic vibration, to the ultrasonic transducer 26, and the supply state of high-frequency electric power to the treatment section 25 and electrode plate). Specifically, the controller 30 exhausts the gas from the suction port 36 to the exhaust source 38 through the exhaust channel 35, in accordance with the transmission of ultrasonic vibration to the treatment section 25 and the supply of high-frequency electric power by the supply of energy from the energy source 28.

In addition, in the energy treatment instrument (insertion instrument) 20, a gas supply channel 41 is formed between an inner peripheral surface of the sheath (shaft section) 21 and an outer peripheral surface of the transmitting member 23, that is, in the inside of the sheath (route forming member) 21. The gas supply channel 41 opens to the outside of the sheath 21 in a jet port 42 at the distal end of the sheath 21. In addition, in the inside of the held section 22, a gas supply tube 43 is connected to the sheath 21. The inside of the gas supply tube 43 communicates with the gas supply channel 41 in a communication position Z1. The gas supply tube 43 extends to the outside of the held section 22 in the outside of the body, and the other end of the gas supply tube 43 is connected to a gas supply source 45 which is disposed in the outside of the body. In the meantime, a gas is prevented from flowing out from the gas supply channel 41 without passing through the inside of the gas supply tube 43 or the jet port 42.

The gas supply source 45 includes a pressure adjusting valve and a gas storage tank. The controller 30 detects the actuation state of the pressure adjusting valve, and controls the actuation of the pressure adjusting valve. Specifically, the controller 30 detects the state of the gas supply by the gas supply source 45, and controls the gas supply by the gas supply source 45. By the pressure adjusting valve being actuated by the controller 30, a gas is supplied from the gas storage tank of the gas supply source 45 to the gas supply channel 41 through the inside of the gas supply tube 43. Thereby, a flow of gas from the proximal side (the outside of the body) toward the distal side (body cavity) is formed in the gas supply channel 41, and the gas from the gas supply source 45 is supplied toward the jet port 42 from the proximal side to the distal side. In addition, the supplied gas is jetted into the body cavity 101 from the jet port 42 which is located at the distal end (in the distal portion) of the sheath (shaft section) 21.

In the present embodiment, the controller 30 controls the gas supply by the gas supply source 45, based on the supply state of energy from the energy source 28 (the supply state of electric power, which generates ultrasonic vibration, to the ultrasonic transducer 26, and the supply state of high-frequency electric power to the treatment section 25 and electrode plate). Specifically, the controller 30 supplies the gas from the gas supply source 45 to the jet port 42 through the gas supply channel 41, in accordance with the transmission of ultrasonic vibration to the treatment section 25 and the supply of high-frequency electric power by the supply of energy from the energy source 28.

In the present embodiment, in the direction parallel to the longitudinal axis L of the sheath 21 (energy treatment instrument 29), an extension dimension d1 from the jet port 42 of the gas supply channel 41 in the sheath 21 to the communication position Z1 with the inside of the gas supply tube 43 is greater than an extension dimension d2 of the hole 33 (from the proximal end to distal end of the hole 33) of the trocar 31. Specifically, the extension dimension (channel extension dimension) d1 along the longitudinal axis L from, the jet port 42 of the gas supply channel 41 toward the proximal side is greater than the extension, dimension (hole extension dimension) d2 of the hole 33 of the trocar 31. Thus, in the state in which the sheath 21 and transmitting member 23 are inserted through the hole 33 of the trocar 31, the jet port 42 of the gas supply channel 41 is located on the distal side with respect to the distal end of the trocar 31 (the distal end of the hole 33), and the communication position Z1 with the inside of the gas supply tube 43 in the gas supply channel 41 is located on the proximal side with respect to the proximal end of the trocar 31 (the proximal end of the hole 33).

Next, the function and advantageous effects of the medical treatment system 1 of the present embodiment will be described. When a treatment is performed by using the medical treatment system 1, the trocars 6 and 31, which are fixing instruments, are fixed to the body wall 100. Then, the insertion section 3 of the endoscope 2 is inserted through the hole 8 of the trocar (first trocar) 6, and the insertion section 3 is inserted into the body cavity 101. In addition, the sheath 21 and transmitting member 23 are inserted through the hole 33 of the trocar (second trocar) 31, and the sheath 21 and transmitting member 23 are inserted into the body cavity 101. Further, the treatment section 25 of the transmitting member 23 is put in contact with a treated target such as a biological tissue, and the treatment is performed. At this time, the insertion section 3 is moved in the body cavity 101 to such a position that the treated region by the treatment section 25 and a vicinity thereof are located in the view field range V of the imaging element 11.

In the treatment, in accordance with the input of the energy operation, energy is supplied from the energy source 28 by the controller 30, vibration generated by the ultrasonic transducer 26 is transmitted to the treatment section 25, and high-frequency electric power is supplied to the treatment section 25 and electrode plate (not shown). The high-frequency electric power is supplied to the treatment section 25, and a high-frequency current flows through the treated target which is in contact with the treatment section 25, and thereby smoke occurs in the treated region by the treatment section 25 and the vicinity thereof (i.e. the view field range V of the imaging element 11). In addition, by the treatment section 25 vibrating due to ultrasonic vibration in the state in which a liquid such as body fluid adheres to the treatment section 25, mist occurs in the treated region by the treatment section 25 and the vicinity thereof.

In the present embodiment, a gas is supplied into the body cavity 101 by the gas supply source 45 in the treatment. The gas from the gas supply source 45 is supplied through the inside of the gas supply tube 43 and the gas supply channel 41, and is jetted into the body cavity 101 from the jet port 42 provided at the distal end of the sheath (shaft section) 21 of the energy treatment instrument (insertion instrument) 20. Here, in the present embodiment, the extension dimension d1 along the longitudinal axis L from the jet port 42 of the gas supply channel 41 toward the proximal side is greater than the extension dimension d2 of the hole 33 of the trocar 31. Thus, in the state in which the sheath 21 and transmitting member 23 are inserted through the hole 33 of the trocar 31, the jet port 42 of the gas supply channel 41 is located on the distal side with respect to the distal end of the trocar 31 (the distal end of the hole 33). Accordingly, in the treatment, the jet port 42 of the gas supply channel 41, compared to the distal end of the trocar 31, becomes less distant from the treated region by the treatment section 25 (the view field range V of the imaging element 11). Specifically, the position of the jet port 42, from which the gas is jetted into the body cavity 101, becomes closer to the treated region by the treatment section 25. Thereby, even if at least one of the smoke and mist is generated in the treated region by the treatment section 25 and the vicinity thereof, the generated smoke and/or mist can properly be eliminated from the treated region and the vicinity thereof (i.e. the view field range V of the imaging element 11) by the gas which is jetted from the jet port 42 of the gas supply channel 41. Since the smoke and/or mist generated from the view field range V of the imaging element 11 is eliminated, it is possible to secure visibility by the imaging element 11 in the treated region of the treatment section 25 and the vicinity of the treated region, and to properly prevent a disturbance of an image of the subject, which is displayed on the monitor 15.

Additionally, since the treatment section 25 projects toward the distal side from the distal end of the sheath 21, where the jet port 42 is located, the jet port 42 opens toward the treated region by the treatment section 25 and the vicinity thereof in the treatment. Thus, the gas supplied through the gas supply channel 41 is jetted from the jet port 42 toward the treated region and the vicinity thereof (i.e. the view field range V of the imaging element 11). By the gas being jetted toward the view field range V of the imaging element 11, the smoke and/or mist generated in the view field range V of the imaging element 11 can easily be eliminated. Thereby, the visibility by the imaging element 11 is further enhanced in the treated region of the treatment section 25 and the vicinity thereof.

Additionally, in the present embodiment, in accordance with the supply of energy (high-frequency electric power and ultrasonic vibration) to the treatment section 25 by the output of energy from the energy source 28, the controller 30 executes control to supply the gas from the gas supply source 45 to the jet port 42 through the gas supply channel 41. Thus, at a timing when at least one of the smoke and mist is generated in the treated region by the treatment section 25 and the vicinity thereof by the high-frequency electric power and ultrasonic vibration supplied (transmitted) to the treatment section 25, the gas is exactly jetted from the jet port 42 of the gas supply channel 41 to the treated region and the vicinity thereof. Therefore, the smoke and/or mist can more exactly be eliminated in the view field range V of the imaging element 11.

Additionally, in the present embodiment, in the treatment, the gas in the body cavity 101 is exhausted to the outside of the body by the exhaust source 38. By the exhaust being performed through the exhaust channel 35 by the exhaust source 38, the gas in the body cavity 101 is sucked into the exhaust channel 35 from the suction port 36 which is located at the distal end of the hole 33 of the trocar 31. In this embodiment, in the treatment, the distal end (jet port 42) of the sheath 21 and the treatment section 25 are located on the distal side with respect to the distal end of the trocar 31. Thus, compared to the jet port 42 of the gas supply channel 41, the suction port 36 of the exhaust channel 35 is located more distant from the treatment section 25. Specifically, in the treatment, in the state in which the suction port 36 is more distant from the treated region by the treatment section 25 than the jet port 42, the gas is jetted from the jet port 42 into the body cavity 101 and the gas is sucked from the body cavity 101 through the suction portion 36. By the simultaneous performance of the jet of the gas from the jet port 42 and the suction of the gas in the suction port 36, a flow of gas is formed in the body cavity 101 from the vicinity of the jet port 42 toward the vicinity of the suction port 36. Accordingly, in the treatment, since the jet port 42 and suction port 36 are located as described above, a flow of gas occurs in the body cavity 101 from the treated region by the treatment section 25 and the vicinity thereof (i.e. the view field range V of the imaging element 11) toward the vicinity of the suction port 36 which is located distant from the treated region. Thereby, the smoke and/or mist generated in the view field range V of the imaging element 11 can easily be eliminated toward the location (suction port 36) which is distant from the treated region. Therefore, in the treated region of the treatment section 25 and the vicinity thereof, the visibility by the imaging element 11 is further enhanced.

Additionally, in the present embodiment, in accordance with the supply of energy (high-frequency electric power and ultrasonic vibration) to the treatment section 25 by the output of energy from the energy source 28, the controller 30 executes control to exhaust the gas from the suction port 36 to the exhaust source 38 through the exhaust channel 35. Thus, at a timing when at least one of the smoke and mist is generated in the treated region by the treatment section 25 and the vicinity thereof by the high-frequency electric power and ultrasonic vibration supplied (transmitted) to the treatment section 25, the flow of gas exactly occurs from the treated region by the treatment section 25 and the vicinity thereof toward the vicinity of the suction port 36. Therefore, the smoke and/or mist can more exactly be eliminated in the view-field range V of the imaging element 11.

(Modifications)

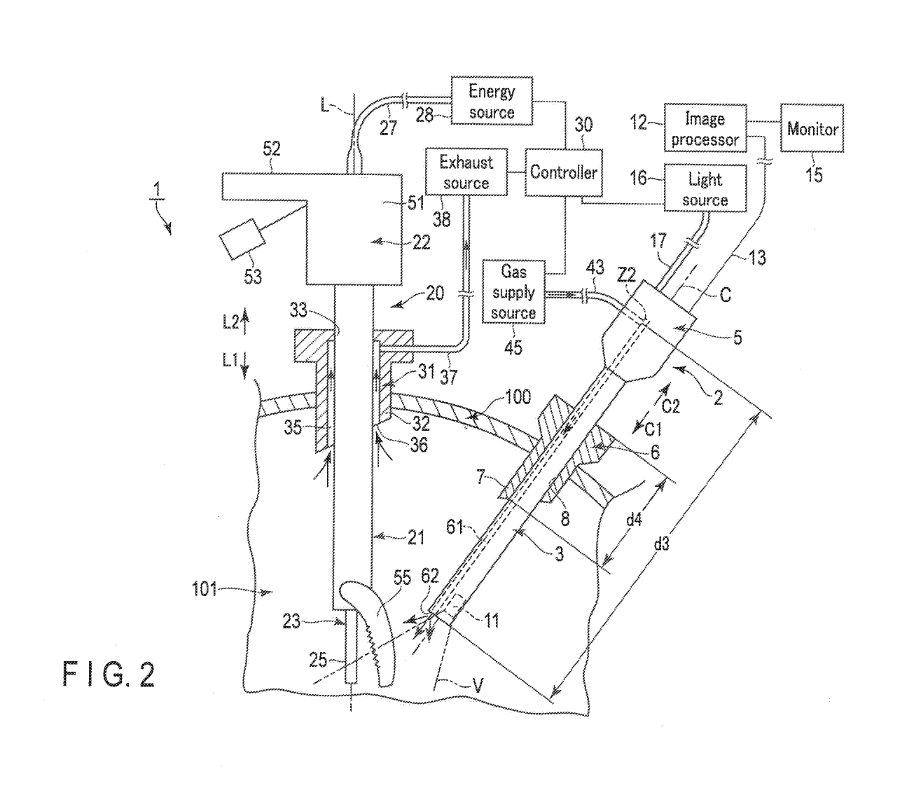

In the meantime, in the first embodiment, the gas supply channel 41, in which the jet port 42 is located in the body cavity 101 in the treatment, is provided in the energy treatment instrument 20 (the inside of the sheath 21), but the restriction to this is unnecessary. For example, in a first modification illustrated in FIG. 2, a gas supply channel 61 extends along the longitudinal axis C of the endoscope (rigid endoscope) 2 in the inside of the insertion section (shaft section) 3 of the endoscope (insertion instrument) 2. Accordingly, in the present modification, the gas supply channel 61, as well as the imaging cable 13 and light guide 17, extends in the inside of the insertion section 3 which is the shaft section (route forming member). In addition, in this modification, the gas supply channel (41) is not provided in the energy treatment instrument 20 which is an insertion member that is a separate body from the endoscope (insertion instrument) 2.

In the present modification, too, the exhaust channel 35 is formed in the trocar (second trocar) 31 through which the energy treatment instrument 20 is inserted. In addition, the gas in the body cavity 101 is sucked into the exhaust channel 35 from the suction port 36 formed at the distal end of the trocar 31, and is exhausted to the exhaust source 38 in the outside of the body through the exhaust channel 35 and the inside of the exhaust tube 37. In this modification, however, a jaw (grasping member) 55 is rotatably attached to the distal portion of the sheath 21, and the jaw 55 is openable and closable relative to the treatment section 25. In addition, in this modification, the held section 22 of the energy treatment instrument 20 includes a held section main body 51 which extends along the longitudinal axis L, a stationary handle (grip) 52 which extends from the held section main body 51 in a direction crossing the longitudinal axis L, and a movable handle (handle) 53 which is openable and closable relative to the stationary handle 52. By closing the movable handle 53 relative to the stationary handle 52, the jaw 55 closes relative to the treatment section 25 of the transmitting member 23. Thereby, a treated target is grasped between the jaw 55 and the treatment section 25. In addition, in the state in which the treated target is grasped, ultrasonic vibration is transmitted to the treatment section 25, and high-frequency electric power is supplied to the treatment section 25 and jaw 55. Specifically, a bipolar treatment, in which the treatment section 25 and jaw 55 function as electrodes of high-frequency electric power, is performed. The treatment section 25 treats the grasped treated target by using the supplied energy (ultrasonic vibration and high-frequency electric power).

In the present modification, the gas supply channel 61 opens to the outside of the insertion section 3 in a jet port 62 of a distal surface of the insertion section 3. In addition, the gas supply tube 43 is connected to the operation section 5 of the endoscope 2. In the operation section 5, the inside of the gas supply tube 43 communicates with the gas supply channel 61 in a communication position Z2. Accordingly, in the present modification, the gas from the gas supply source 45 is supplied through the inside of the gas supply tube 43 and the gas supply channel 61, and is supplied toward the jet port 62 from the proximal side to the distal side in the gas supply channel 61. Further, the supplied gas is jetted in the direction of the longitudinal axis C from the jet port 62 toward the body cavity 101. In the present modification, an extension dimension (channel extension dimension) d3 along the longitudinal axis L from the jet port 62 of the gas supply channel 61 toward the proximal side is greater than an extension dimension (hole extension dimension) d4 of the hole 8 of the trocar (first trocar) 6 through which the insertion section 3 is inserted. Thus, in the state in which the insertion section 3 is inserted through the hole 8 of the trocar 6, the jet port 62 of the gas supply channel 61 is located on the distal side with respect to the distal end of the trocar 6 (the distal end of the hole 8), and the communication position Z2 with the inside of the gas supply tube 43 in the gas supply channel 61 is located on the proximal side with respect to the proximal end of the trocar 6 (the proximal end of the hole 8).

In the treatment in this modification, too, the insertion section 3 is moved in the body cavity 101 to such a position that the treated region by the treatment section 25 and the vicinity thereof are located in the view field range V of the imaging element 11. At this time, since the extension dimension d3 along the longitudinal axis C from the jet port 62 of the gas supply channel 61 toward the proximal side is greater than the extension dimension d4 of the hole 8 of the trocar 6, the jet port 62 of the gas supply channel 61 is located on the distal side with respect to the distal end of the trocar 6 (the distal end of the hole 8). Accordingly, in the treatment, the jet port 62 of the gas supply channel 61, compared to the distal end of the trocar 6, becomes less distant from the treated region by the treatment section 25 (the view field range V of the imaging element 11). Specifically, the position of the jet port 62, from which the gas is jetted into the body cavity 101, becomes closer to the treated region by the treatment section 25. Thereby, even if at least one of smoke and mist is generated in the treated region by the treatment section 25 and the vicinity thereof, the generated smoke and/or mist can properly be eliminated from the treated region and the vicinity thereof (i.e. the view field range V of the imaging element 11) by the gas which is jetted from the jet port 62 of the gas supply channel 61.

In addition, the imaging element 11 performs imaging in the distal direction of the insertion section 3 as the imaging direction. Besides, the gas is jetted from the jet port 62 toward the distal side of the insertion section 3. Accordingly, in the present modification, too, in the treatment, the gas supplied through the gas supply channel 61 is jetted from the jet port 62 toward the treated region and the vicinity thereof (i.e. the view field range V of the imaging element 11).

Besides, in the treatment, since the treatment section 25 is disposed in the view field range V of the imaging element 11, the jet port 62 of the gas supply channel 61 is located near the treatment section 25 and jaw 55. On the other hand, in the treatment, since the treatment, section 25 and jaw 55 are located on the distal side with respect to the distal end of the trocar 31, the suction port 36, which is formed sit the distal end of the trocar 31, is located distant from the treatment section 25. Accordingly, in this modification, too, in the treatment, the suction port 36 of the exhaust channel 35 is more distant from the treatment section 25 than the jet port 62 of the gas supply channel 61. Thus, in the present modification, too, in the body cavity 101 at the time of the treatment, a flow of gas is formed from the treated region by the treatment section 25 and the vicinity thereof (i.e. the view field range V of the imaging element 11) toward the vicinity of the suction port 36 which is located distant from the treated region.

Since the treatment is performed as described above, the same function and advantageous effects as in the first embodiment can be obtained in the present modification.

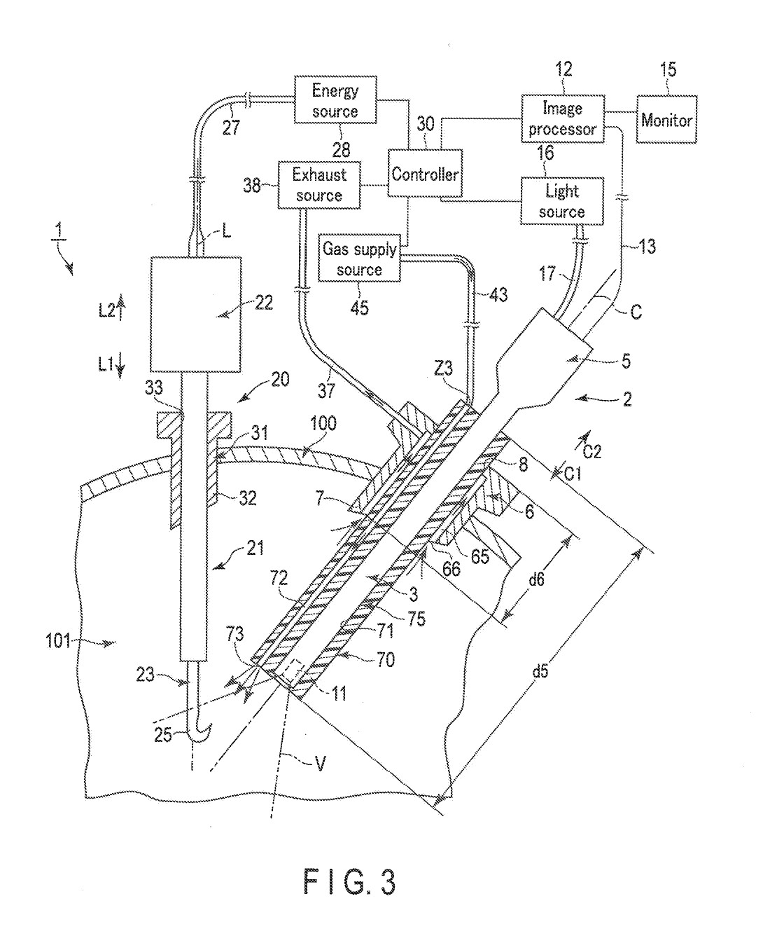

Additionally, for example, in a second modification as illustrated in FIG. 3, there is provided an overtube 70 through which the insertion section 3 of the endoscope 2 is inserted. A gas supply channel 72 extends through a tube main body (shaft section) 75 of the overtube (insertion instrument) 70. Besides, in this modification, an insertion channel 71, which is in a separate state from the gas supply channel 72, extends through the tube main body (route forming member) 75 of the overtube 70. The insertion channel 71 does not communicate with the gas supply channel 72. By the insertion section 3 of the endoscope (rigid endoscope) 2 being inserted through the insertion channel 71, the overtube 70 is attached to the insertion section 3. At this time, the distal end of the insertion section 3 is located in the inside of the insertion channel 71 (tube main body 75), and the insertion section 3 does not project from a distal surface of the overtube 70 toward the distal side. In the state in which the overtube 70 is attached to the insertion section 3, the insertion channel 71 and gas supply channel 72 extend from the proximal side toward the distal side along the longitudinal axis C of the endoscope 2 (substantially in parallel to the longitudinal axis C). In addition, in the state in which the overtube 70 is attached to the insertion section 3, the imaging element 11 of the insertion section 3 is located in a distal portion of the insertion channel 71 (a distal portion of the overtube 70). In the present modification, the endoscope 2 and the energy treatment instrument 20, which are insertion members separate from the overtube (insertion instrument) 70, are not provided with gas supply channels (61: 41).

In the present modification, an exhaust channel (35) is not formed in the trocar (second trocar) 31 through which the energy treatment instrument 20 is inserted. In addition, in this modification, the treatment section 25 of the energy treatment instrument 20 is formed in a hook shape. In the treatment, the treatment section 25 is hooked on the treated target. In addition, the treatment section 25 treats the hooked treated target by using the supplied energy (high-frequency electric power and ultrasonic vibration).

In the present modification, in the state in which the overtube 70 is attached to the insertion section 3 of the endoscope 2, the overtube 70 is inserted through the hole 8 of the trocar 6. Thereby, the insertion section 3 of the endoscope 2 and the tube main body 75 of the overtube 70 are inserted into the body cavity 101.

Besides, in the present modification, an exhaust channel 65 is formed by the hole 8 of the trocar (first trocar) 6 through which the overtube 70 and the insertion section 3 of the endoscope 2 are inserted. In addition, a suction port 66 of the exhaust channel 65 is formed at the distal end of the trocar 6. One end of the exhaust tube 37 is connected to the trocar 6, and the inside of the exhaust tube 37 communicates with the exhaust channel 65. In the meantime, in the state in which the overtube 70 (tube main body 75) and the insertion section 3 of the endoscope 2 are inserted through the hole 8 of the trocar 6, airtightness is kept between the overtube 70 and trocar 6 at the proximal end of the hole 8. Thus, a gas is prevented from flowing out from the exhaust channel 65, without passing; through the inside of the exhaust tube 37 or the suction port 66. In the present modification, the gas in the body cavity 101 is sucked into the exhaust channel 65 from the suction port 66 formed at the distal end of the trocar 6, and is exhausted to the exhaust source 38 in the outside of the body through the exhaust channel 65 and the inside of the exhaust tube 37.

In the present embodiment, the gas supply channel 72 opens to the outside of the overtube 70 in a jet port 73 of the distal surface of the overtube 70 (tube main body 75). In addition, the gas supply tube 43 is connected to a proximal surface of the overtube 70. Besides, the inside of the gas supply tube 43 communicates with the gas supply channel 72 in a communication position Z3 of the proximal surface of the overtube 70. Accordingly, in the present modification, the gas from the gas supply source 45 is supplied through the inside of the gas supply tube 43 and the gas supply channel 72, and is supplied toward the jet port 73 from the proximal side to the distal side in the gas supply channel 72. Further, the supplied gas is jetted from the jet port 73 toward the body cavity 101. In the present modification, an extension dimension (channel extension dimension) d5 along the longitudinal axis C of the endoscope 2 from the jet port 73 of the gas supply channel 72 toward the proximal side is greater than an extension dimension (hole extension dimension) d6 of the hole 8 of the trocar (first trocar) 6 through which the overtube 70 is inserted. Thus, in the state in which the overtube 70 and the insertion section 3 are inserted through the hole 8 of the trocar 6, the jet port 73 of the gas supply channel 72 is located on the distal side with respect to the distal end of the trocar 6 (the distal end of the hole 8), and the communication position Z3 with the inside of the gas supply tube 43 in the gas supply channel 72 is located on the proximal side with respect to the proximal end of the trocar 6 (the proximal end of the hole 8).

In the treatment in this modification, by moving the tube main body 75 of the overtube 70 in the body cavity 101, the insertion section 3 is moved to such a position that the treated region by the treatment section 25 and the vicinity thereof are located in the view field range V of the imaging element 11. At this time, since the extension dimension d5 along the longitudinal axis C from the jet port 73 of the gets supply channel 72 toward the proximal side is greater than the extension dimension do of the hole 8 of the trocar 6, the jet port 73 of the gas supply channel 72 is located on the distal side with respect to the distal end of the trocar 6 (the distal end of the hole 8). In addition, by the tube main body 75 and insertion section 3 being inserted through the hole 8 of the trocar 6, the imaging element 11 is also located on the distal side with respect to the distal end of the trocar 6. Accordingly, in the treatment, the jet port 73 of the gas supply channel 72, compared to the distal end of the trocar 6, becomes less distant, from the treated region by the treatment section 25 (the view field range V of the imaging element 11). Specifically, the position of the jet port 73, from which the gas is jetted into the body cavity 101, becomes closer to the treated region by the treatment section 25. Thereby, even if at least one of smoke and mist is generated in the treated region by the treatment section 25 and the vicinity thereof, the generated smoke and/or mist can properly be eliminated from the treated region and the vicinity thereof (i.e. the view field range V of the imaging element 11) by the gas which is jetted from the jet port 73 of the gas supply channel 72.

Additionally, in this modification, too, the imaging element 11 performs imaging in the distal direction of the insertion section 3 (the distal direction of the overtube 70) as the imaging direction. Besides, the gas is jetted from the jet port 73 toward the distal side of the insertion section 3. Accordingly, in the present modification, too, the gas supplied through the gas supply channel 72 is jetted from the jet port 73 toward the treated region and the vicinity thereof (i.e. the view field range V of the imaging element 11).

Besides, in the treatment, since the imaging element 11 is located in the distal portion of the overtube 70, and the treatment section 25 is disposed in the view field range V of the imaging element 11, the jet port 73 of the gas supply channel 72, which is formed on the distal surface of the overtube 70, is located near the treatment section 25. On the other hand, in the treatment, since the imaging element 11 and jet port 73 are located on the distal side with respect to the distal end of the trocar 6, the suction port 66, which is formed at the distal end of the trocar 6, is located distant from the treatment section 25. Accordingly, in this modification, too, in the treatment, the suction port 66 of the exhaust channel 65 is located more distant from the treatment section 25 than the jet port 73 of the gas supply channel 72. Thus, in the present modification, too, in the body cavity 101 at the time of the treatment, a flow of gas is formed from the treated region by the treatment section 25 and the vicinity thereof (i.e. the view field range V of the imaging element 11) toward the vicinity of the suction port 66 which is located distant from the treated region.

Since the treatment is performed as described above, the same function and advantageous effects as in the first embodiment can be obtained in the present modification.

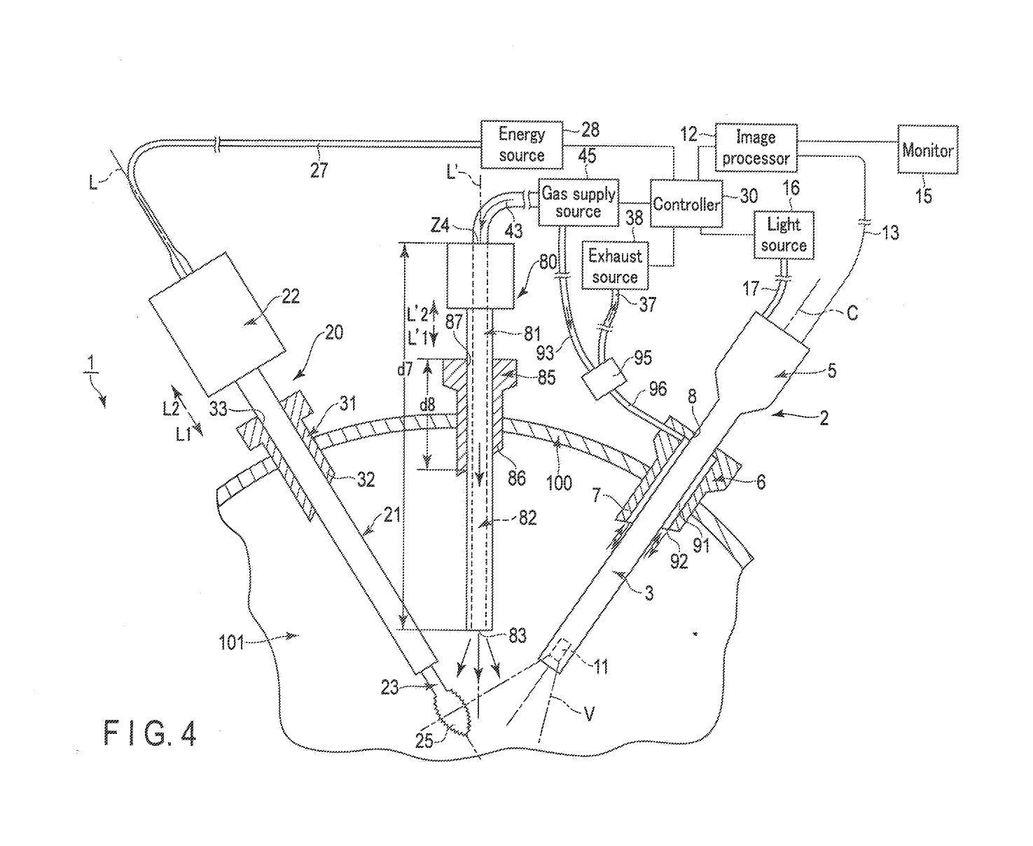

Additionally, for example, in a third modification as illustrated in FIG. 4, an assistant tool (insertion instrument) 80, which is inserted into the body cavity 101, is provided separately from the energy treatment instrument 20 and endoscope 2. A gas supply channel 82 extends through an assistant tool insertion section (shaft section) 81 of the assistant tool (insertion instrument) 80. The assistant tool 80 has a longitudinal axis (assistant tool longitudinal axis) L′. Here, one side of two directions parallel to the longitudinal axis L′ of the assistant tool 80 is a distal side of the assistant tool 80 (arrow L′1 side in FIG. 4), and a side opposite to the distal side is a proximal side of the assistant tool 80 (arrow L′2 side in FIG. 4). In the assistant tool 80, the assistant tool insertion section (route forming member) 81 extends along the longitudinal axis L′. The assistant tool insertion section 81 has a distal portion and a proximal portion, and a distal end of the assistant tool 80 is formed by a distal end of the assistant tool insertion section 81.

In the present modification, there is provided a trocar (third trocar) 85 as a fixing instrument, separately from the trocar (first trocar) 6 through which the insertion section of the endoscope 2 is inserted, and the trocar (second trocar) 31 through which the sheath 21 of the energy treatment instrument 20 is inserted. The trocar 85 includes a piercing portion (third piercing portion) 86. By the piercing portion 86 being pierced into the body wall 100, the trocar 85 is fixed to the body wall 100. Besides, a hole (third hole) 87 is formed in the trocar 85. In the state in which the trocar 85 is fixed to the body wall 100, the outside of the body (external environment) and the body cavity 101 communicate with each other through the hole 87. The assistant tool insertion section 81 of the assistant tool 80 is inserted through the hole 87 of the trocar 85 from the distal side, and is inserted into the body cavity 101. In the state in which the assistant tool insertion section 81 is inserted in the body cavity 101, the distal portion of the assistant tool insertion section 81 is located on the distal side (distal direction side) with respect to a distal end of the trocar 85 (a distal end of the piercing portion 87). Specifically, the assistant tool insertion section 81 projects from the distal end of the trocar 85 toward the distal side.

In the present modification, the endoscope 2 and energy treatment instrument 20, which are insertion members separate from the assistant tool (insertion instrument) 80, are not provided with gas supply channels (61: 41). In addition, in the present modification, an exhaust channel (35) is not formed in the trocar (second trocar) 31 through which the energy treatment instrument 20 is inserted. Besides, in this modification, the treatment section 25 of the energy treatment instrument 20 is formed in a blade shape. In this modification, too, the treatment section 25 treats the treated target by using the supplied energy (high-frequency electric power and ultrasonic vibration).

In the present modification, a common channel 91 is formed by the hole 8 of the trocar (first trocar) 6 through which the insertion section 3 of the endoscope 2 is inserted. In addition, a common opening 92 of the common channel 91 is formed at the distal end of the trocar 6. One end of a common tube 96 is connected to the trocar 6, and the inside of the common tube 96 communicates with the common channel 91. In the meantime, in the state in which the insertion section 3 of the endoscope 2 is inserted through the hole 8 of the trocar 6, airtightness is kept between the insertion section 3 and the trocar 6 at the proximal end of the hole 8. Thus, a gas is prevented from flowing out from the common channel 91, without passing through the inside of the common tube 96 or the common opening 92.

The other end of the common tube 96 is connected to a change-over valve 95. The exhaust tube 37 is connected to the change-over valve 95, and one end of a sub-gas supply tube 93, which is a separate body from the gas supply tube 43, is also connected to the change-over valve 95. The other end of the sub-gas supply tube 93 is connected to the gas supply source 45. In the present modification, the gas supply source 45 can supply a gas through the inside of the gas supply tube 43, and can also supply a gas through the inside of the sub-gas supply tube 93. In addition, in this modification, the change-over valve 95 can be switched, by a surgeon's operation or the like, between a state in which the change-over valve 95 establishes communication between the inside of the exhaust tube 37 and the inside of the common tube 96, and a state in which the change-over valve 95 establishes communication between the inside of the sub-gas supply tube 93 and the inside of the common tube 96.

In the state in which the inside of the exhaust tube 37 and the inside of the common tube 96 communicate with each other in the change-over valve 95, the gas in the body cavity 101 is sucked into the common channel 91 from the common opening 92 which is formed at the distal end of the trocar 6, and the gas is exhausted to the exhaust source 38 located in the outside of the body through the common channel 91, the inside of the common tube 96 and the inside of the exhaust tube 37. On the other hand, in the state in which the inside of the sub-gas supply tube 93 and the inside of the common tube 96 communicate with each other in the change-over valve 95, the gats from the gas supply source 45 is supplied to the common channel 91 through the inside of the sub-gas supply tube 93 and the inside of the common tube 96. In addition, the gas is supplied from the proximal side to the distal side in the common channel 91, and the supplied gas is jetted into the body cavity 101 from the common opening 92 which is formed at the distal end of the trocar 6. Accordingly, in the present modification, the common channel 91 can be commonly used as the gas supply channel which supplies the gas into the body cavity 101, and as the exhaust channel which exhausts the gas from the body cavity 101. In addition, the common opening 92 can be commonly used as the jet port which jets out the gas into the body cavity 101, and as the suction port which sucks the gas from the body cavity 101.

In the present modification, the gas supply channel 82 opens to the outside of the assistant tool 80 in the jet port 83 of the distal surface of the assistant tool insertion section 81 (assistant tool 80). In addition, in the assistant tool 80, the gas supply tube 43 is connected, and the inside of the gas supply tube 43 communicates with the gas supply channel 82 in a communication position Z4. Accordingly, in the present modification, the gas, which is supplied from the gas supply source 45 to the inside of the gas supply tube 43, passes through the gas supply channel 82, and is supplied toward the jet port 83 in the gas supply channel 82 from the proximal side to the distal side. In addition, the supplied gas is jetted from the jet port 83 toward the body cavity 101. In this modification, an extension dimension (channel extension dimension) 61 along the longitudinal axis L′ of the assistant tool 80 from the jet port 83 of the gas supply channel 82 toward the proximal side is greater than an extension dimension (hole extension dimension) d8 of the hole 87 of the trocar (third trocar) 85 through which the assistant tool 80 is inserted. Thus, in the state in which the assistant tool 80 is inserted through the hole 87 of the trocar 85, the jet port 83 of the gas supply channel 82 is located on the distal side with respect to the distal end of the trocar 85 (the distal end of the hole 87), and the communication position Z4 with the inside of the gas supply tube 43 in the gas supply channel 82 is located on the proximal side with respect to the proximal end of the trocar 85 (the proximal end of the hole 87).

In the treatment in this modification, in the body cavity 101, the insertion section 3 is moved to such a position that the treated region by the treatment section 25 and the vicinity thereof are located in the view field range V of the imaging element 11. In addition, the extension dimension d7 along the longitudinal axis L′ from the jet port 83 of the gas supply channel 82 toward the proximal side is greater than the extension dimension d8 of the hole 87 of the trocar 85. Thus, by inserting the assistant tool insertion section 81 through the hole 87 of the trocar 85, the jet port 83 of the gas supply channel 82 is located on the distal side with respect to the distal end of the trocar 85 (the distal end of the hole 87). Accordingly, in the treatment, the jet port 83 of the gas supply channel 82, compared to the distal end of the trocar 85, becomes less distant from the treated region by the treatment section 25 (the view field range V of the imaging element 11). Specifically, the position of the jet port 83, from which the gas is jetted into the body cavity 101, becomes closer to the treated region by the treatment section 25. Thereby, even if at least one of smoke and mist is generated in the treated region by the treatment section 25 and the vicinity thereof, the generated smoke and/or mist can properly be eliminated from the treated region and the vicinity thereof (i.e. the view field range V of the imaging element 11) by the gas which is jetted from the jet port 83 of the gas supply channel 82.

Additionally, in the present modification, too, the imaging element 11 performs imaging in the distal direction of the insertion section 3 as the imaging direction, and the treated region by the treatment section 25 and the vicinity thereof become the view field range V of the imaging element 11. In addition, in this modification, the gas, which is supplied through the gas supply channel 82, is jetted from the jet port 83 to the distal side toward the treated region and the vicinity thereof.

Besides, in the treatment, since the gas is jetted from the jet port 83 toward the treated region and the vicinity thereof, the jet port 33 of the gas supply channel 82, which is formed on the distal surface of the assistant tool 80, is located near the treatment section 25. On the other hand, in the treatment, since the imaging element 11 is located on the distill side with respect to the distal end of the trocar 6, the common opening 92, which is formed at the distal end of the trocar 6, is located distant from the treatment section 25. Accordingly, in this modification, in the treatment, the common opening 92 of the common channel 91 is located more distant from the treatment section 25 than the jet port 83 of the gas supply channel 82. Thus, in the present modification, in the body cavity 101 at the time of the treatment, by performing exhaust through the common channel 91, a flow of gas is formed from the treated region by the treatment section 25 and the vicinity thereof (i.e. the view field range V of the imaging element 11) toward the vicinity of the common opening 92 which is located distant from the treated region.

Since the treatment is performed as described above, the same function and advantageous effects as in the first embodiment can be obtained in the present modification.

In the meantime, the shape of the treatment section 25 and the aspect of the treatment by the treatment section 25 are not limited to those in the above-described embodiment, etc. In addition, in the above-described embodiment, the treatment section 25 treats the treated target by using the high-frequency electric power and ultrasonic vibration as energy. However, the restriction to this is unnecessary. For example, the energy treatment instrument 20 may be provided with a heating body, and the treatment section 25 may perform a treatment by using, as energy, heat which the heating body generates. Specifically, it should suffice if the treatment section 25, which performs a treatment by using energy, is provided in the distal portion of the energy treatment instrument 20. Besides, the shape of the treatment section 25 can be changed as needed.

Additionally, in the first embodiment, the gas supply channel 41 is formed between the sheath 21 and transmitting member 23 of the energy treatment instrument 20. However, in one modification, the gas supply channel (41) may be formed along the longitudinal axis L in the inside of the transmitting member (shaft section) 23. In this case, the transmitting member (shaft section) 23 is formed to be hollow, and the jet port (42) of the gas supply channel (41) is formed in the treatment section 25 of the transmitting member 23.

Additionally, if such configurations are adopted that the gas supply channel (41; 61; 72; 82) is provided in one insertion instrument (20; 2; 70; 80) which is inserted into the body cavity 101, and the extension dimension (d1; d3; d5; d1) of the gas supply channel (41; 61; 72; 82) along the longitudinal axis (L; C; L′) from the jet port (43; 62; 73; 83) toward the proximal side is greater than the extension dimension (d2; d4; d6; d8) of the hole (33; 8; 87) of the fixing instrument (31; 6; 85) through which the insertion instrument (20; 2; 70; 80) is inserted, parts of the configurations of the above-described first embodiment and the first to third modifications may be combined with parts of configurations of other embodiments, etc. For example, in one modification, like the first embodiment, the gas supply channel (41) is provided in the energy treatment instrument (20). However, the exhaust channel (35) is not formed in the trocar (31) through which the energy treatment instrument (20) is inserted. Like the second modification, the exhaust channel (65) is formed in the trocar (6) through which the insertion section (3) of the endoscope (2) is inserted. In addition, in another modification, like the third modification, the gas supply channel (82) is provided in the assistant tool (80). However, the common channel (91) is not formed in the trocar (6) through which the endoscope (2) is inserted, and, like the first modification, the exhaust channel (35) is formed in the trocar (31) through which the energy treatment instrument (20) is inserted. In each of these modifications, the same function and advantageous effects as in the above-described embodiment, etc. can be obtained. Besides, the combination between the gas supply channel and the exhaust channel (common channel) can be changed as needed.

In the above-described embodiment, etc., an insertion instrument (20; 2; 70; 80), which is inserted into a body cavity (101) by being inserted through a hole (33; 8; 87) of a fixing instrument (31; 6; 85), is provided in a medical treatment system (1). The medical treatment system (1) includes the fixing instrument. (31; 6; 85) configured to form the hole (33; 8; 87) which establishes communication between an outside of a body and the body cavity (101), and configured to be fixed to a body wall (100), and also includes a treatment section (25) configured to perform a treatment by using energy in the body cavity (101). In addition, the insertion instrument (20; 2; 70; 80) includes a shaft section (21; 23; 3; 75; 81) which extends along a longitudinal axis (L; C; L′) from a proximal portion toward a distal portion, and projects, by being inserted, through the hole (33; 8; 87) of the fixing instrument (31; 6; 85), from a distal end of the fixing instrument (31; 6; 85) toward a distal side in the body cavity (101). A gas supply channel (41; 61; 72; 82) is formed in the shaft section (21; 23; 3; 75; 81), and the gas supply channel (41; 61; 72; 82) has a jet port (42; 62; 73; 83), which jets a gas, in the distal portion of the shaft section (21; 23; 3; 75; 81). In the gas supply channel (41; 61; 72; 82), the gas is supplied from a proximal side to the distal side toward the jet port (42; 62; 73; 83). An extension dimension (d1; d3; d5; d7) along the longitudinal axis (L; C; L′) of the gas supply channel (41; 61; 72; 82) from the jet port (42; 62; 73; 83) toward the proximal side is greater than an extension dimension (d2; d4; d6; d8) of the hole (33; 8; 87) of the fixing instrument (31; 6; 85).

Hereinafter, a characteristic item will additionally be described.

(Additional Item 1)

A route forming member which is inserted into a body cavity by being inserted through a hole of a fixing instrument, the fixing instrument being configured to form the hole which establishes communication between an outside of a body and the body cavity, and configured to be fixed to a body wall,

the route forming member having a gas supply channel which establishes communication between the outside of the body and the body cavity, and being disposed in a state in which a jet port of the gas supply channel is located on an inner side in the body cavity with respect to an end of the fixing instrument on the inner side in the body cavity.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1-12. (canceled)

13. A medical treatment system comprising:
an endoscope including an insertion section which extends along a longitudinal axis and is inserted into a body cavity, the endoscope including a distal portion, and an observation window provided in the distal portion and configured to observe a subject, and the endoscope further including a gas supply channel which is passed through an inside of the insertion section and has a jet port configured to jet a gas in a direction along the longitudinal axis;
a first trocar having a first hole which keeps airtightness with the insertion section of the endoscope in a state in which the insertion section of the endoscope is inserted through the first hole, the first trocar being configured such that the distal portion of the endoscope projects from a distal end thereof;
a treatment instrument including a treatment section configured to perform a treatment of a treated target by using energy;
a second trocar having a second hole through which the treatment instrument is inserted, and an exhaust channel configured to exhaust the jetted gas from the body cavity to an outside, the second trocar being configured such that the treatment section of the treatment instruments projects from a distal end thereof;
a gas supply tube connected to the gas supply channel of the endoscope; and
a gas supply source to which the gas supply tube is connected, the gas supply source being configured to perform pressure adjustment and to supply the gas to the gas supply channel via the gas supply tube.

14. The medical treatment system of claim 13, further comprising:
an exhaust tube connected to the exhaust channel in the second trocar; and
an exhaust source which includes an exhaust pump, and to which the exhaust tube is connected, the exhaust source being configured to exhaust a gas via the exhaust channel and the exhaust tube, by the exhaust pump being driven.

15. The medical treatment system of claim 14, further comprising:
an energy source configured to supply energy to the treatment instrument; and
a controller configured to control supply of the energy from the energy source to the treatment instrument,
wherein the controller is configured to control exhaust by the exhaust source in accordance with the supply of the energy from the energy source.

16. The medical treatment system of claim 13, further comprising:
an energy source configured to supply energy to the treatment instrument; and
a controller configured to control supply of the energy from the energy source to the treatment instrument,
wherein the controller is configured to control gas supply by the gas supply source in accordance with the supply of the energy from the energy source.

17. The medical treatment system of claim 13, further comprising:
an exhaust tube connected to the exhaust channel in the second trocar;
an exhaust source which includes an exhaust pump, and to which the exhaust tube is connected, the exhaust source being configured to exhaust a gas via the exhaust channel and the exhaust tube, by the exhaust pump being driven;
an energy source configured to supply energy to the treatment instrument; and
a controller configured to control supply of the energy from the energy source to the treatment instrument,
wherein the controller is configured to execute control to supply the gas from the gas supply source into the body cavity, and to execute control to exhaust the gas, which is supplied from the gas supply source, to an outside of a body, in accordance with the supply of the energy from the energy source.

18. The medical treatment system of claim 13, wherein the treatment section is configured to perform the treatment by using at least one of ultrasonic vibration and high-frequency electric power as the energy.

19. An endoscope comprising:
an insertion section including a distal surface and extending along a longitudinal axis;
an observation window provided on the distal surface and configured to observe a subject;
a gas supply channel provided in the insertion section; and
a jet port which opens to an outside of the insertion section on the distal surface of the insertion section, the jet port being configured to jet a gas, which is supplied through the gas supply channel, in a direction along the longitudinal axis.

20. A medical treatment system comprising:
an endoscope including an insertion section which extends along a longitudinal axis and is inserted into a body cavity;
a treatment instrument including a treatment section which is configured to perform a treatment of a treated target by using energy;
a plurality of trocars through each of which the endoscope or the treatment instrument is inserted, each of the trocars having a hole through which the endoscope or the treatment instrument is inserted, at least one of the trocars includes an exhaust channel configured to exhaust a gas in the body cavity to an outside; and
a gas supply source including a gas storage tank, and configured to jet a gas, via a gas supply tube, toward a treated region of the treatment instrument from a jet port which is located on a distal side with respect to a distal end of the trocar.

21. The medical treatment system of claim 20, wherein the jet port is provided in the treatment instrument.
